(19) United States
(12) Patent Application Publication
(10) Pub. No.: US 2020/0062809 A1
(43) Pub. Date: Feb. 27, 2020

(54) METHOD FOR SEPARATING AND PURIFYING MUSSEL ADHESIVE PROTEIN

(71) Applicant: KOLLODIS BIOSCIENCES, INC., North Augusta, SC (US)

(72) Inventors: Sang Jae LEE, Seoul (KR); Bong Jin HONG, Pohang-si, Gyeongsangbuk-do (KR)

(73) Assignee: KOLLODIS BIOSCIENCES, INC., North Augusta, SC (US)

(21) Appl. No.: 16/495,446

(22) PCT Filed: Mar. 20, 2017
(86) PCT No.: PCT/KR2017/002980

§ 371 Date: Sep. 19, 2019

(51) Int. Cl. C07K 14/435; C07K 1/30; C07K 1/36; C07K 7/08

(52) U.S. Cl. CPC C07K 14/43509; C07K 7/08; C07K 1/36; C07K 1/30

(57) ABSTRACT

Provided is a method for separating and purifying a mussel adhesive protein, including the steps of: (1) crushing cells containing a mussel adhesive protein; (2) centrifuging the crushed cells to obtain an insoluble protein aggregate including the mussel adhesive protein; (3) treating the insoluble protein aggregate with an acidic organic solvent to obtain a low-purity mussel adhesive protein solution; (4) selectively precipitating the mussel adhesive protein by controlling the acidity of the low-purity mussel adhesive protein solution; and (5) treating the precipitate with a surfactant to remove endotoxins from the mussel adhesive protein. The method of the subject matter can purify a large amount of mussel adhesive proteins of high purity with a simple process. In particular, the subject matter can be applied effectively to the development of novel uses of mussel adhesive proteins by significantly reducing production costs through economical production of the mussel adhesive protein.

### TECHNICAL FIELD

The present invention relates to a method for separating and purifying a mussel adhesive protein with high purity. More particularly, the present invention relates to a method of economically and effectively separating a physiologically functional adhesive protein with high purity by treating a mussel adhesive protein produced by a fermentation process as well as mussel adhesive protein comprising a physiologically functional peptide (such as an extracellular matrix-derived peptide, an antibacterial peptide, and the like) with a proper solvent and controlling the acidity of a mussel adhesive protein solution.

### BACKGROUND ART

A mussel adhesive protein exhibits a strong adhesive characteristic in water because a large amount of 3,4-dihydroxyl-L-alanine (DOPA) is included in the mussel adhesive protein. The mussel adhesive protein having such characteristic exhibits strong adhesive strength in water, and also exhibits strong adhesive strength to surfaces of various materials such as plastics, glass, metals, Teflon, and the like. The adhesive strength of the mussel adhesive protein in water still remains to be solved in the field of chemical adhesives. Also, because the mussel adhesive protein is known to be biocompatible without attacking human cells or causing any immune response, the mussel adhesive protein is highly applicable in the field of medicine and health care such as adhesion of biological tissues during surgery or adhesion of broken teeth (D. R. Filpula, et al., Biotechnol. Prog. 6, 171-177, 1990).

Technology for mass-producing the mussel adhesive protein in Escherichia coli (E. coli) by means of DNA recombination technology has been successfully developed, and some types of the technology (for example, a nickel ion-chromatography method using nickel ions, a method using an isoelectric point, and the like) have been put into practice as technology for separating and purifying the mussel adhesive protein. However, a method for separating and purifying a mussel adhesive protein having a predetermined high purity is not established yet (Korean Patent Laid-Open Publication Nos. KR 10-08680470000, and KR 10-08728470000; J. Porath, et al., Biochemistry 22, 1621-1630, 1983; P. Z. OFarrell, et al., Cell 12, 1133-1142, 1977).

A separation/purification procedure provided in the related art will be described in further detail, as follows. Cultured E. coli cells are centrifuged, and then homogenized on ice at 200 W for 10 seconds using a cell homogenizer (for example, a sonicator, and the like) to obtain an inclusion body including the mussel adhesive protein. The mussel adhesive protein present in the inclusion body is selectively extracted from an acetic acid solution. To remove E. coli-derived impurities (for example, proteins, lipids, and the like) included in the mussel adhesive protein after the primary extraction, the method includes a process of charging a chromatography column with nickel ions to separate a protein by means of the histidine-nickel ion affinity. Also, a separation/purification method using the isoelectric point includes a process of primarily extracting a mussel adhesive protein from the acetic acid solution, followed by selectively precipitating the mussel adhesive protein using various acids and bases. In addition, the separation/purification process using the nickel ion chromatography provided in the related art has limitations in medical applications because it is very expensive, and histidine inducing an inflammatory response is also included in the mussel adhesive protein (W. D. Won, et al., Appl. Environ. Microbiol. 31, 576-580, 1976).

Accordingly, the present invention provides technology for separating and purifying a mussel adhesive protein with high purity under the control of acidity using an isoelectric point of the mussel adhesive protein in a separation/purification process which does not include a separation/purification process using chromatography according to the affinity of histidine.

### DISCLOSURE

### Technical Problem

The present invention is designed to solve the problems of the prior art, and therefore it is an object of the present invention to provide a method capable of removing impurities from the various recombinant mussel adhesive proteins under the control of acidity, thereby obtaining a mussel adhesive proteins with a high purity of 90% or more. Also, it is another object of the present invention to provide a separation/purification method capable of obtaining mussel adhesive proteins with high purity regardless of molecular weights and structures of the mussel adhesive proteins.

### Technical Solution

To solve the above problems, according to an aspect of the present invention, there is provided a method for separating and purifying a mussel adhesive protein, which includes: (1) homogenizing cells containing a mussel adhesive protein; (2) centrifuging the homogenate to obtain an insoluble protein aggregate (i.e., an inclusion body) including the mussel adhesive protein; (3) treating the insoluble protein aggregate with an acidic organic solvent to obtain a low-purity mussel adhesive protein solution; (4) selectively precipitating the mussel adhesive protein under the control of acidity of the low-purity mussel adhesive protein solution; and (5) treating the precipitate with a surfactant to remove endotoxins from the mussel adhesive protein.

According to one embodiment of the present invention, E. coli, yeast, animal cells, and the like may be used as the cells of the step (1) without any limitation, but the present invention is not limited thereto.

According to one embodiment of the present invention, the cells of the step (1) may be stirred with a lysis buffer, and may be then homogenized using a high-pressure homogenizer, but the present invention is not limited thereto.

According to one embodiment of the present invention, the acidic organic solvent of the step (3) may have a pH value ranging from pH 1 to 6, but the present invention is not limited thereto.

According to another embodiment of the present invention, a conventional acidic solution such as acetic acid, citric acid, lactic acid may be used as the acidic organic solvent of the step (3), but the present invention is not limited thereto.

According to one preferred embodiment of the present invention, the acetic acid may be 5 to 40% (v/v), preferably 20 to 30% (v/v) acetic acid, but the present invention is not limited thereto.

According to one embodiment of the present invention, isoelectric points (pI) of protein impurities and an isoelectric point of the mussel adhesive protein are used under the control of acidity to selectively precipitate the mussel adhesive protein of the step (4), but the present invention is not limited thereto.

According to another embodiment of the present invention, the control of acidity may be carried out by adding 9 to 11 N NaOH, preferably 10 N NaOH to the mussel adhesive protein solution to increase the acidity of the solution to pH 11 to 14, preferably pH 12 to 13, and more preferably pH 12.8, centrifuging the mussel adhesive protein solution to collect a supernatant, and adding acetic acid to the supernatant to neutralize and titrate the acidity of the solution to pH 6 to 7, but the present invention is not limited thereto.

According to one embodiment of the present invention, the mussel adhesive protein may have a peptide sequence selected from the group consisting of SEQ ID NO: 1 to SEQ ID NO: 21, but the present invention is not limited thereto.

According to another embodiment of the present invention, a functional peptide selected from the group consisting of an extracellular matrix, a growth factor, an anticancer peptide, and an antibacterial peptide may be fused to the C-terminus or N-terminus of the mussel adhesive protein.

According to one preferred embodiment of the present invention, the antibacterial peptide may have a peptide sequence selected from the group consisting of SEQ ID NO: 27 to SEQ ID NO: 30, or SEQ ID NO: 56 to SEQ ID NO: 59, but the present invention is not limited thereto.

### Advantageous Effects

According to the present invention, the method of the present invention, which is characterized by including a process for separating and purifying a mussel adhesive protein using an acidic organic solvent under the control of acidity, can purify a large amount of the mussel adhesive protein with high purity using a simple process. In particular, the present invention can be effectively applied to development of novel uses of the mussel adhesive protein by significantly reducing production costs through economical production of the mussel adhesive protein.

### DESCRIPTION OF DRAWINGS

The above and other features and advantages of the present invention will become more apparent to those of ordinary skill in the art by describing in detail exemplary embodiments thereof with reference to the attached drawing, in which:

FIG. 1 is a diagram of a separation/purification process using acetic acid and the control of acidity.

FIGS. 2A to 2C show the SDS PAGE results of separated and purified mussel adhesive proteins having different molecular weights. FIG. 2A shows the batch results for separating and purifying proteins having a molecular weight of 12 kDa and 23 kDa, FIG. 2B shows the batch result of six time separation and purification of a protein having a high molecular weight of 38 kDa, which are identical to the results for separation and purification of the protein without any batch collection, and FIG. 2C shows that the separated and purified protein exhibits lot-to-lot consistency.

FIG. 3 shows the results of separating and purifying a mussel adhesive protein whose isoelectric point somewhat increases by attachment of a biologically active peptide. Comparing the results for separating and purifying a protein to which an extracellular matrix (e.g., a fibronectin peptide, an antibacterial peptide or the like) is attached and a protein to which a functional peptide is not attached, it is revealed that there is a slight difference in the control of acidity during a separation/purification procedure, but the protein may be generally separated and purified with high purity regardless of the type of peptides using the technology of the present invention.

FIG. 4 shows an immunofluorescence image and a graph showing similar CYP450 activities in a surface coated with a mussel adhesive protein including a collagen-derived GFPGER peptide and a surface coated with collagen.

FIG. 5 shows the results of antibacterial activities of an antibacterial adhesive protein fused with an antibacterial peptide (KLWKKWAKKWLKLWKA; SEQ ID NO: 27) against E. coli.

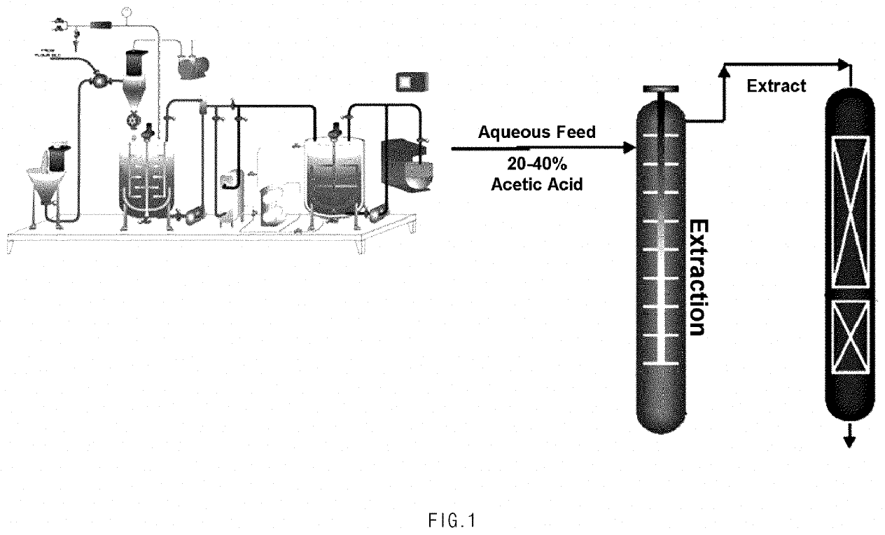

### BEST MODE

The present invention provides a method for separating and purifying a mussel adhesive protein, which includes:

(1) homogenizing E. coli including a mussel adhesive protein;

(2) centrifuging the homogenate to obtain an insoluble protein aggregate including the mussel adhesive protein;

(3) treating the insoluble protein aggregate with an acidic organic solvent to obtain a low-purity mussel adhesive protein solution;

(4) selectively precipitating the mussel adhesive protein under the control of the acidity of the low-purity mussel adhesive protein solution; and

(5) treating the precipitate with a surfactant to remove endotoxins from the mussel adhesive protein.

According to one embodiment of the present invention, because a recombinant mussel adhesive protein which is expressed in microbial cells (e.g., E. coli, yeast, or the like) or animal cells is expressed in a water-soluble and/or water-insoluble form of a transformant, respective separation and purification methods may vary depending on an expression pattern. When the mussel adhesive protein or derivatives thereof are expressed in a water-soluble form, a recombinant protein may be purified by subjecting a supernatant of the cell homogenate to chromatography using a column filled with an affinity resin, for example, a nickel resin. Also, when the mussel adhesive protein is expressed in a water-insoluble form, cell by-products (pellets) of the cell homogenate may be suspected in an acidic organic solvent, preferably a conventional acidic organic solvent having pH 1 to 6 to prepare a suspension, and the suspension may be then centrifuged to separate a supernatant. However, because a low-purity mussel adhesive protein having a purity of 50 to 70% is obtained in this way, an additional purification process is needed as described above in the present invention.

In the step (1), the cells may be stirred with a lysis buffer, and then homogenized using a high-pressure homogenizer, but the present invention is not limited thereto.

In the step (3), examples of the acidic organic solvent that may be used herein include conventional acidic organic solvents such as acetic acid, citric acid, lactic acid, and the like, but the present invention is not limited thereto. In the case of the acetic acid, 5 to 40% (v/v) acetic acid may be used. Preferably, the cell by-products (pellets) may be more effectively dissolved in a 20 to 30% (v/v) acetic acid solution.

The isoelectric points (pI) of the protein impurities and the isoelectric point of the mussel adhesive protein are properly used under the control of the acidity to selectively precipitate the mussel adhesive protein as the key point of the last step. The isoelectric point of the mussel adhesive protein is approximately 10.8. In this case, the isoelectric point of the mussel adhesive protein may somewhat increase when a biologically active group or a certain amino acid group is introduced for the purpose of other certain physicochemical functions. The isoelectric points of the mussel adhesive proteins into which various biologically active peptides are introduced are summarized in Table 1.

TABLE 1
Isoelectric points of physiologically functional
mussel adhesive proteins

| Biologically | SEQ | Molecular | |
|---|---|---|---|
| active | ID | weight | Isoelectric |
| peptides | NO | (Dalton) | point |
| SPPRRARVT | 22 | 24624.08 | 9.96 |
| TWYKIAFQRNRK | 23 | 25195.76 | 9.95 |
| KNSFMALYLSGRLVFALG | 24 | 25605.34 | 9.91 |
| TAGSCLRKFSTM | 25 | 24886.42 | 9.91 |
| GLPGER | 31 | 24245.65 | 9.89 |
| KGHRGF | 33 | 24285.67 | 9.93 |
| DGEA | 34 | 24246.59 | 9.90 |
| GEFYFDLRLKGDK | 35 | 25172.67 | 9.87 |
| TAIPSCPEGTVPLYS | 36 | 25119.62 | 9.85 |
| RQVFQVAYIIIKA | 37 | 25133.77 | 9.92 |
| IKVAV | 38 | 24113.57 | 9.91 |
| NRWHSIYIRFG | 39 | 25134.63 | 9.93 |
| RKRLQVQLSIRT | 40 | 25082.68 | 9.97 |
| RYVVLPR | 41 | 24486.98 | 9.93 |
| YIGSR | 42 | 24179.54 | 9.91 |
| KAFDITYVRLKF | 43 | 25085.68 | 9.91 |
| RNIAEIIKDI | 44 | 24769.28 | 9.89 |
| KLDAPT | 45 | 24228.61 | 9.89 |
| RGD | 46 | 23931.22 | 9.90 |
| GRGDSP | 47 | 24172.47 | 9.90 |
| WQPPRARI | 48 | 24608.08 | 9.94 |
| KNNQKSEPLIGRKKT | 49 | 25325.9 | 9.94 |
| REDV | 50 | 24102.41 | 9.88 |

According to one embodiment of the present invention, an acidity control process for selective precipitation of the mussel adhesive protein is as follows. 9 to 11 N NaOH, and preferably 10 N NaOH is added to the mussel adhesive protein solution to increase the acidity (pH) of the solution to pH 12 to 13, preferably approximately pH 12.8, and then centrifuged to collect a supernatant. After acetic acid is added to the supernatant to neutralize and titrate the acidity (pH) of the solution to pH 6 to 7, the mussel adhesive protein obtained by centrifugation may be dissolved in a proper amount of purified water, and freeze-dried to obtain a mussel adhesive protein having a purity of 90% or more. An acidic solution, for example, acetic acid may be added to the collected solution to neutralize and titrate the acidity of the acidity (pH) of the solution to pH 5 to 6, and the mussel adhesive protein may be diluted with a proper amount of purified water, desalted, and then freeze-dried to obtain a mussel adhesive protein having a purity of 95% or more.

According to one embodiment of the present invention, the present invention provides a separation/purification process capable of obtaining a recombinant mussel adhesive protein having a molecular weight of 12 kDa with a high purity of 90% or more.

According to another embodiment of the present invention, the present invention provides a separation/purification process capable of obtaining a recombinant mussel adhesive protein having a molecular weight of 22.6 kDa with a high purity of 90% or more.

According to still another embodiment of the present invention, the present invention provides a separation/purification process capable of obtaining a recombinant mussel adhesive protein having a molecular weight of 37.8 kDa with a high purity of 90% or more.

According to yet another embodiment of the present invention, the present invention provides a separation/purification process capable of obtaining each of an extracellular matrix mimetic (i.e., a mussel adhesive protein MAPTrix™ ECM) in which a peptide derived from the extracellular matrix is introduced into the carboxyl (C)-terminus or amino (N)-terminus of a mussel adhesive protein having a molecular weight of 22.6 kDa, an antibacterial adhesive in which an antibacterial peptide is introduced into the mussel adhesive protein, and MAPTrix™ GF in which a growth factor is introduced into the mussel adhesive protein with a high purity of 90% or more.

Hereinafter, the present invention will be described in detail.

In the present invention, the mussel adhesive protein is an adhesive protein derived from Mytilus coruscus. In this case, the mussel adhesive protein is preferably a recombinant mussel adhesive protein, but the present invention is not limited thereto. Preferably, the mussel adhesive protein may include any mussel adhesive proteins as disclosed in International Publication No. WO 2006/107183A1 or WO 2005/092920, but the present invention is not limited thereto.

MAPTrix™ provided in one embodiment of the present invention is a mussel adhesive protein functionalized by genetic recombination. In the present invention, the mussel adhesive protein may be used intact, or may be used as a fusion protein to which a first peptide and a second peptide is fused, wherein the first peptide corresponds to the C-terminus or N-terminus or both termini of foot protein 3 (FP-3) set forth in SEQ ID NO: 5, 6, 7 or 8, FP-5 set forth in SEQ ID NO: 10, 11, 12 or 13, or FP-6 set forth in SEQ ID NO: 14, and the second peptide includes one or more selected from the group consisting of mussel adhesive proteins FP-1 (SEQ ID NO: 1), FP-2 (SEQ ID NO: 4), and FP-4 (SEQ ID NO: 9), and fragments of the proteins. Preferably, the first peptide is FP-5 including an amino acid sequence set forth in SEQ ID NO: 10, 11, 12 or 13, and the second peptide is FP-1 including an amino acid sequence set forth in SEQ ID NO: 1, 2 or 3. According to one embodiment of the present invention, the mussel adhesive protein preferably has an amino acid sequence selected from the group consisting of SEQ ID NO: 1 to SEQ ID NO: 21, but the present invention is not limited thereto.

Preferably, the mussel adhesive protein may also be (a) a polypeptide having an amino acid set forth in SEQ ID NO: 4, (b) a polypeptide having an amino acid sequence set forth in SEQ ID NO: 5, (c) a polypeptide in which an amino acid sequences set forth in SEQ ID NO: 6 are repeatedly ligated 1 to 10 times in a sequential manner, and (d) a polypeptide formed by fusion of one or more selected from the group consisting of the polypeptide of (a), the polypeptide of (b) and the polypeptide of (c). The polypeptide of (c) may be preferably a polypeptide having an amino acid sequence set forth in SEQ ID NO: 7, but the present invention is not limited thereto. Also, the fused polypeptide of (d) may be preferably a polypeptide having an amino acid sequence set forth in SEQ ID NO: 1 or SEQ ID NO: 3, but the present invention is not limited thereto.

In the present invention, mutants of the mussel adhesive protein may preferably include polypeptides, which have an additional sequence at the carboxyl terminus (C-terminus) or the amino terminus (N-terminus) of the mussel adhesive protein or have some amino acids substituted with other amino acids, on the assumption that the adhesive strength of the mussel adhesive protein is maintained intact. More preferably, the mutants may include polypeptides in which a polypeptide consisting of 3 to 25 amino acids, which include a physiologically functional peptide, for example, RGD, is linked to the carboxyl terminus or amino terminus of the mussel adhesive protein, or in which 1 to 100%, preferably 5 to 100%, and more preferably 50 to 100% of the total number of tyrosine residues constituting the mussel adhesive protein is substituted with 3,4-dihydroxyphenyl-L-alanine (DOPA).

The mussel adhesive protein according to the present invention may be preferably mass-produced by a genetic engineering method by inserting a foreign gene into a conventional vector constructed for the purpose of expressing the foreign gene, but the present invention is not limited thereto. The vector may be properly selected according to the type and characteristic of the host cells, or constructed de novo to produce the protein. A method of transforming host cells with the vector, and a method of producing a recombinant protein from the transformant may be easily performed using conventional methods. Methods such as selection and construction of the aforementioned vector, transformation with the vector, and expression of a recombinant protein may be easily performed by those skilled in the art, and some modifications made to the conventional methods are also encompassed in the present invention.

MAPTrix™ provided in the present invention is a mussel adhesive protein functionalized by genetic recombination. An extracellular matrix, a growth factor, and a functional peptide having an antibacterial or anticancer function may be added to the C-terminus, the N-terminus, or both termini of the mussel adhesive protein, or added between hybrid mussel adhesive proteins by means of DNA recombination technology. For example, a functional peptide may be added between FP-1 and FP-5 in the case of a fusion protein FP-151 having a structure in which one FP-5 is linked between two FP-1s. Also, different functional peptides may be added between the both termini or between the fusion proteins. In the present invention, any peptides that may be naturally occurring or may be artificially synthesized may be used as the functional peptide fused to the adhesive protein without any limitation. For example, a biologically active peptide serving as the functional peptide is a naturally occurring or synthesized peptide that is derived from an extracellular matrix protein to mimic biochemical or biophysical signals of a naturally occurring extracellular matrix. The extracellular matrix protein may be a fibrous protein such as collagen, fibronectin, laminin, vitronectin, and the like. For example, a collagen-derived peptide GFPGER (SEQ ID NO: 32) may be added to the carboxyl (C)-terminus or between FP-1 and FP-5, and a laminin-derived peptide IKVAV (SEQ ID NO: 38) may be added to the amino (N)-terminus.

As another functional peptide, a biologically active peptide derived from a growth factor, which is associated with the regulation of various physiological processes including development, regeneration, and wound repair, may be added. For example, the mussel adhesive proteins functionalized with peptides (set forth in SEQ ID NO: 51 and SEQ ID NO: 52) derived from an acidic fibroblast growth factor, and peptides (set forth in SEQ ID NO: 53 to SEQ ID NO: 55) derived from a basic fibroblast growth factor are fibroblast growth factor mimetics that have activities similar to the naturally occurring or recombinant fibroblast growth factors.

The antibacterial peptide included in the antibacterial adhesive provided as one example in the present invention may be added to the carboxyl (C)-terminus, the amino (N)-terminus, or both termini of the mussel adhesive protein, or added between hybrid mussel adhesive proteins by means of DNA recombination technology. For example, an antibacterial peptide may be added between FP-1 and FP-5 in the case of the fusion protein FP-151. Also, the antibacterial peptide may be added between the both termini or between the fusion proteins. For example, the antibacterial peptide may include an antibacterial peptide such as magainin or dermaseptin, which is an α-helical peptide of 23 amino acids isolated from the skin of an African clawed frog (Xenopus laevis), and may be an antibacterial peptide such as human defensin, cathelicidin LL-37, histatin, and the like, but the present invention is not limited thereto.

According to one embodiment of the present invention, all types of peptides that are naturally occurring or artificially synthesized may be used as the antibacterial peptide fused to the adhesive protein according to the present invention. The antibacterial peptide exerts an antibacterial effect by means of a pathway for destroying cell membranes of a microorganism or penetrating the cell membranes to inhibit the metabolic functions. In the present invention, all types of the antibacterial peptides exerting an antibacterial effect are included in the pathway for destroying the cell membranes of the microorganism.

Preferably, the antibacterial peptide to be fused to the adhesive protein may be selected from the antibacterial peptide having an antibacterial effect on gram-negative bacteria as well as gram-positive bacteria. More preferably, the antibacterial peptide may be selected from KLWKKWAKKWLKLWKA (SEQ ID NO: 27), FALALKALKKL (SEQ ID NO: 28), ILRWPWWPWRRK (SEQ ID NO: 29), AKRHHGYKRKFH (SEQ ID NO: 30), KWKLFKKIGAVLKVL (SEQ ID NO: 56), LVKLVAGIKKFLKWK (SEQ ID NO: 57), IWSILAPLGTTLVKLVAGIGQQKRK (SEQ ID NO: 58), GTNNWWQSPSIQN (SEQ ID NO: 59).

According to one embodiment of the present invention, an antibacterial coating film may also be prepared by coating a polystyrene film with urethane acrylate including the antibacterial adhesive protein and optically curing the polystyrene film. According to one preferred embodiment of the present invention, the antibacterial activity of the coating film may be determined by determining a bacterial reduction rate of gram-negative bacteria, for example, E. coli, on the uncoated and coated surfaces.

The following examples are provided to describe the preferred embodiment of the present invention. However, it should be understood that the following certain examples are given for the purpose of illustration of the present invention only, and are not intended to limit the scope of the present invention. As described in the present invention, it will be apparent that functionally identical articles, compositions, and methods fall within the scope of the present invention.

### Example 1: Construction of Expression Vector with Mussel Adhesive Protein

To prepare mussel adhesive fusion proteins having various molecular weights, the mussel adhesive fusion proteins set forth in SEQ ID NO: 3 (FP1), SEQ ID NO: 15 (FP151) and SEQ ID NO: 21 (13151) was designed, and requested to NovaCell Technology Inc. to construct expression vectors, respectively. E. coli BL21 (DE3) was transformed with the tailored vectors.

### Example 2: Construction of Expression Vector with Functional Mussel Adhesive Protein

To prepare mussel adhesive fusion proteins having various functionalities, fusion peptides in which conventional functional peptide sequences set forth in SEQ ID NO: 22 to SEQ ID NO: 30 are linked to the C-terminal or N-terminal regions of a mussel adhesive protein, and requested to NovaCell Technology Inc. to construct expression vectors, respectively. E. coli BL21 (DE3) was transformed with the tailored vectors. In this case, the added sequences are listed in Table 2. Hereinafter, the antibacterial peptide-fused mussel proteins set forth in SEQ ID NO: 27 to SEQ ID NO: 30 are represented by “A,” “B,” “C,” and “D,” respectively.

TABLE 2

| | SEQ | |
|---|---|---|
| Added peptide | ID | Fusion site in mussel |
| sequence | NO | adhesive protein |
| SPPRRARVT | 22 | C-terminus |
| TWYKIAFQRNRK | 23 | C-terminus |
| KNSFMALYLSKG | 24 | C-terminus |
| GFPGER | 32 | C-terminus |
| FRHRNRKGY | 26 | C-terminus |
| KLWKKWAKKWLKLWKA | 27 | C-terminus |
| FALALKALKKL | 28 | N-terminus |
| ILRWPWWPWRRK | 29 | C-terminus |
| AKRHHGYKRKFH | 30 | C-terminus |

### Example 3: Preparation of Various Mussel Adhesive Protein Derivatives

3.1: Culture of E. coli BL21 (DE3)

E. coli BL21 (DE3) was cultured in an LB medium (5 g/liter yeast extract, 10 g/liter Tryptone, and 10 g/liter NaCl), and IPTG was added at a final concentration of 1 mM to induce expression of the recombinant antibacterial peptide-fused mussel adhesive protein until the optical density of a culture broth at 600 nm reached approximately 0.6. The E. coli BL21 (DE3) culture broth was centrifuged at 13,000 rpm and 4° C. for 10 minutes to obtain cell pellets, which were stored at −80° C.

3.2: Confirmation of Expression of Mussel Adhesive Protein

The cell pellets were diluted with 100 μg of a buffer solution for SDS-PAGE (0.5 M Tris-HCl, pH 6.8, 10% glycerol, 5% SDS, 5% β-mercaptoethanol, and 0.25% bromophenol blue), and boiled at 100° C. for 5 minutes so that the cell pellets were denatured. For SDS-PAGE, a sample was electrophoresed in a 15% SDS-polyacrylamide gel, and then stained with a Coomasie blue stain to detect and check a protein band.

### Example 4: Separation and Purification of Mussel Adhesive Protein

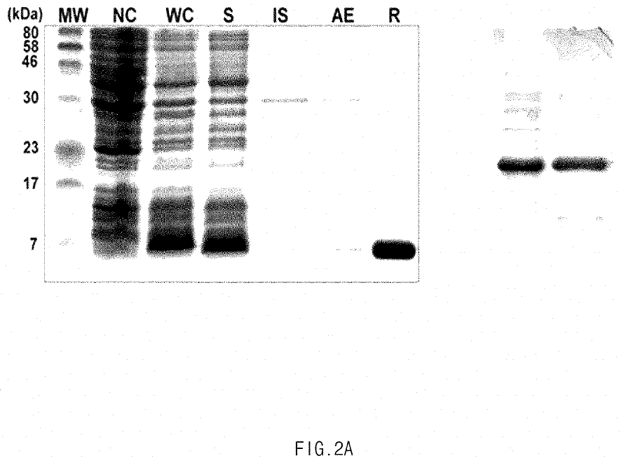

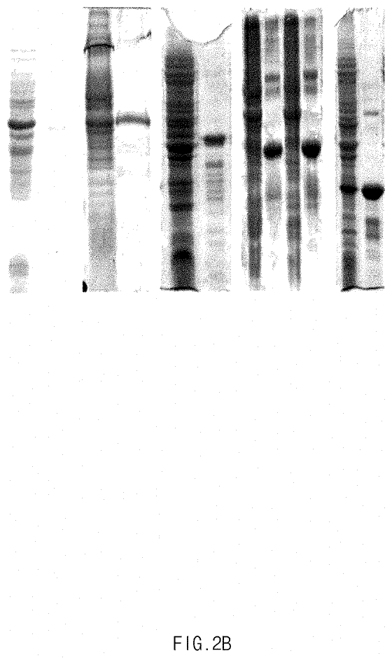

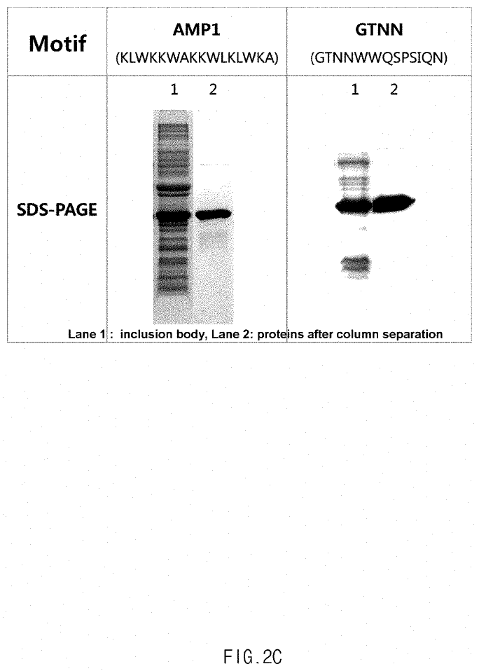

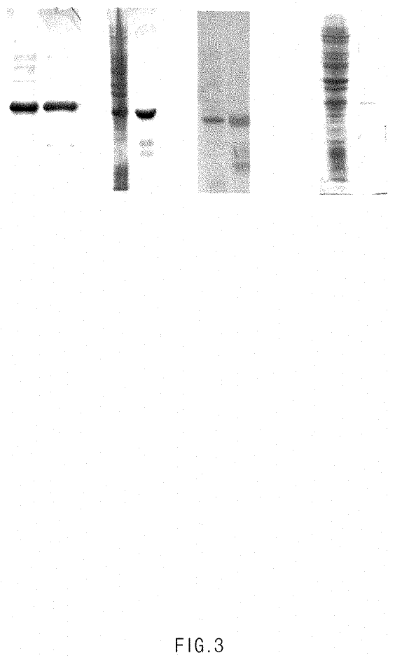

The cell pellets obtained in Example 3.1 were stirred in a lysis buffer (2.4 g/L sodium phosphate monobasic, 5.6 g/L sodium phosphate dibasic, 10 mM EDTA, and 1% Triton X-100), and homogenized using a high-pressure homogenizer. The homogenate was centrifuged at 9,000 rpm for 20 minutes to obtain an insoluble protein aggregate including the mussel adhesive protein. The antibacterial peptide-fused mussel adhesive protein was extracted from the insoluble protein aggregate using 25% acetic acid, and then centrifuged at 9,000 rpm for 20 minutes to obtain a supernatant including the mussel adhesive protein. Acetone was added to the collected supernatant at a volume 2 to 3 folds higher than that of the supernatant, uniformly mixed for 30 minutes, and then centrifuged at 6,000 rpm for 20 minutes to collect an aggregate including the mussel adhesive protein. The aggregate was dissolved in purified water, and then centrifuged at 9,000 rpm for 20 minutes to collect the mussel adhesive protein uniformly dispersed in deionized water. The pH of the collected supernatant increased to pH 12.8 using 10 N NaOH, and centrifuged under the same conditions to obtain a supernatant. The supernatant was neutralized and titrated to pH 6 to 7 using acetic acid, and then centrifuged under the same conditions to obtain a precipitate of the antibacterial peptide-fused mussel adhesive protein. The obtained precipitate was dissolved in a proper amount of purified water, and then freeze-dried to obtain a lyophilisate of the antibacterial peptide-fused mussel adhesive protein having a purity of 90% or more (FIGS. 2A to 2C and 3). From the SDS results, it can be seen that the purification technology of the present invention was applicable to obtain high-purity mussel adhesive proteins without using expensive chromatography in spite of the molecular weight or the presence of the functional peptide.

### Example 5: Cell Culture Test of Extracellular Matrix-Functional Peptide

The mussel adhesive protein including a peptide GFPGER (SEQ ID NO: 32) derived from collagen serving as one of the extracellular matrixes was coated on a 12-well plate. A coating solution of the mussel adhesive protein was prepared at a concentration of 0.06 mg/mL by dissolving the mussel adhesive protein is distilled water, and each of the wells was coated for an hour by spraying 1.2 mL of the coating solution per well. Thereafter, human-derived liver cells were cultured for 48 hours in the wells coated with the mussel adhesive protein, and the wells coated with collagen (collagen type I, BD Biosciences). As the human normal liver cell line, a Chang cell line (ATCC cat # CCL-13, USA) was used as the cell line used in this experiment. Then, Dulbecco's modified essential medium (DMEM, Gibco, USA) [symbol] 2% FBS (Gibco), penicillin (100 units/mL, Sigma, USA), streptomycin n (100 g/mL, Sigma), and sodium bicarbonate (3.7 g/L, Sigma) were added to each well, and the Chang cell line was cultured at 37° C. in a 5% CO₂ incubator.

Next, the liver cells were collected, homogenized, and then centrifuged at 4° C. and 12,000 rpm for 10 minutes to collect a supernatant. Thereafter, the supernatant was electrophoresed in 10% SDS-PAGE to separate proteins. The separated proteins were washed twice with TBS-T for 10 minutes, and an antigen/antibody reaction was induced at 4° C. using, as primary antibodies, an anti-CYP450 antibody (Chemicon, USA) and an anti-GAPDH antibody (Santacruz, USA), both of which had been diluted 1:1,000 with TBS-T supplemented with 0.5% BSA. Thereafter, the proteins were washed twice with TBS-T for 10 minutes, and incubated at room temperature for an hour using, as a secondary antibody, HRP-conjugated anti-rabbit and anti-mouse IgG (Santacruz) diluted 1:2,000 with TBS-T supplemented with 0.5% BSA. Then, an expression pattern of the CYP450 protein was analyzed.

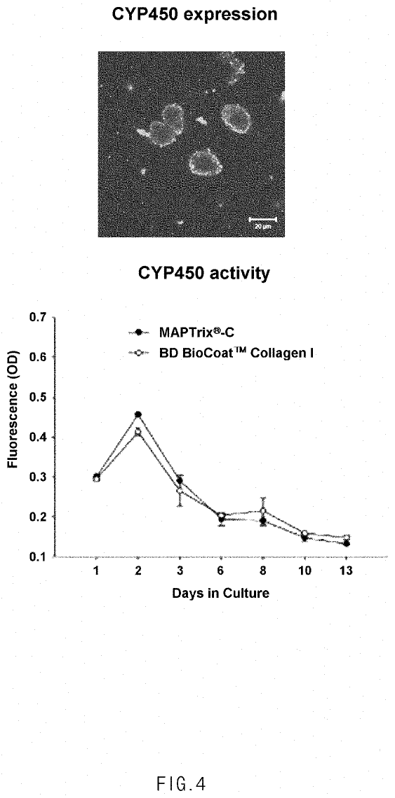

As a result, it can be seen that the protein exhibited similar CYP450 activities on the surface coated with the mussel adhesive protein including GFPGER and the collagen-coated surface (FIG. 4), indicating that the liver cells were normally cultured on the surface coated with the mussel adhesive protein.

### Example 6: Antibacterial Activity Test of Antibacterial Peptide-Fused Mussel Protein

First of all, the antibacterial peptide-fused mussel proteins A, B, C, D were prepared with different concentrations. The concentration for antibacterial activity tests was in a range of 10 to 0.01 mg/mL, and prepared using a phosphate buffered saline (PBS) buffer solution. As the strain for antibacterial activity tests, a gram-negative strain, that is, E. coli was used. E. coli was cultured at 37° C. and 150 rpm in an LB medium while stirring until the optical density of the solution reached 1.0. At the optical density of 1.0, the E. coli culture broth was diluted with PBS until the cells reached 10⁴ CFU/mL. Thereafter, the diluted culture broth was mixed with a ready-made antibacterial peptide fusion protein at a ratio of 9:1 in sterile tubes, and the resulting mixture was incubated at a temperature of 37° C. for an hour in a thermohygrostat. After an hour, 100 μL of the E. coli culture broth was taken from each of the tubes, spread on an agar medium, and then incubated for 24 hours under the same conditions.

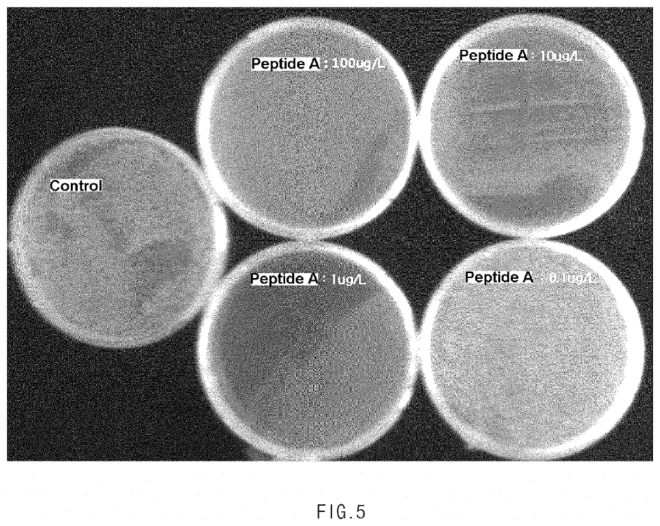

As a result, it was revealed that the antibacterial peptide-fused mussel protein, particularly the antibacterial protein to which the antibacterial peptide of SEQ ID NO: 27 was fused, had an antibacterial activity of 99.99%, compared to the control (FIG. 5).

1. A method for separating and purifying a mussel adhesive protein, comprising the steps of:
(1) homogenizing cells containing a mussel adhesive protein;
(2) centrifuging the homogenate to obtain an insoluble protein aggregate comprising the mussel adhesive protein;
(3) treating the insoluble protein aggregate with an acidic organic solvent to obtain a low-purity mussel adhesive protein solution;
(4) selectively precipitating the mussel adhesive protein under the control of the acidity of the low-purity mussel adhesive protein solution; and
(5) treating the precipitate with a surfactant to remove endotoxins from the mussel adhesive protein.

2. The method of claim 1, wherein the cells of the step (1) are selected from the group consisting of E. coli, yeast, and animal cells.

3. The method of claim 1, wherein the cells of the step (1) are stirred with a lysis buffer, and then homogenized using a high-pressure homogenizer.

4. The method of claim 1, wherein the acidic organic solvent of the step (3) has a pH value ranging from pH 1 to 6.

5. The method of claim 1, wherein the acidic organic solvent of the step (3) is selected from the group consisting of acetic acid, citric acid, and lactic acid.

6. The method of claim 5, wherein the acetic acid is 5 to 40% (v/v) acetic acid.

7. The method of claim 6, wherein the acetic acid is 20 to 30% (v/v) acetic acid.

8. The method of claim 1, wherein isoelectric points (pI) of protein impurities and an isoelectric point of the mussel adhesive protein are used under the control of acidity to selectively precipitate the mussel adhesive protein of the step (4).

9. The method of claim 8, wherein the control of acidity is carried out by adding 9 to 11 N NaOH to the mussel adhesive protein solution to increase the acidity of the solution to pH 12 to 13, centrifuging the mussel adhesive protein solution to collect a supernatant, and adding acetic acid to the supernatant to neutralize and titrate the acidity of the solution to pH 6 to 7.

10. The method of claim 9, wherein the control of acidity is carried out by adding 10 N NaOH to the mussel adhesive protein solution to increase the acidity of the solution to pH 12.8, centrifuging the mussel adhesive protein solution to collect a supernatant, and adding acetic acid to the supernatant to neutralize and titrate the acidity of the solution to pH 6 to 7.

11. The method of claim 1, wherein the mussel adhesive protein has a peptide sequence selected from the group consisting of SEQ ID NO: 1 to SEQ ID NO: 21.

12. The method of claim 1, wherein a functional peptide selected from the group consisting of an extracellular matrix, a growth factor, an anticancer peptide, and an antibacterial peptide is fused to the C-terminus or N-terminus of the mussel adhesive protein.

13. The method of claim 1, wherein an antibacterial peptide is fused to the C-terminus or N-terminus of the mussel adhesive protein.

14. The method of claim 13, wherein the antibacterial peptide has a peptide sequence selected from the group consisting of SEQ ID NO: 27 to SEQ ID NO: 30 and SEQ ID NO: 56 to SEQ ID NO: 59.

15. The method of claim 4, wherein the acidic organic solvent of the step (3) is selected from the group consisting of acetic acid, citric acid, and lactic acid.


SEQUENCE LISTING (59 sequences)

SEQ ID NO: 1 - protein, 10 aa, Artificial Sequence
  model peptide of the tandem repeat decapeptide derived from foot protein 1 (FP-1, Mytilus edulis)
AKPSYPPTYK

SEQ ID NO: 2 - protein, 20 aa, Artificial Sequence
  2 times repeated sequence derived from foot protein 1 (FP-1, Mytilus edulis)
AKPSYPPTYKAKPSYPPTYK

SEQ ID NO: 3 - protein, 60 aa, Artificial Sequence
  6 times repeated sequence derived from foot protein 1 (FP-1, Mytilus edulis)
AKPSYPPTYKAKPSYPPTYKAKPSYPPTYKAKPSYPPTYKAKPSYPPTYKAKPSYPPTYK

SEQ ID NO: 4 - protein, 39 aa, Artificial Sequence
  partial sequence of foot protein type 2 (FP-2, Mytilus californianus)
EVHACKPNPCKNNGRCYPDGKTGYKCKCVGGYSGPTCAC

SEQ ID NO: 5 - protein, 52 aa, Artificial Sequence
  Foot protein type 3 (FP-3, Mytilus edulis)
ADYYGPKYGPPRRYGGGNYNRYGGSRRYGGYKGWNNGWKRGRWGRKYYEFEF

SEQ ID NO: 6 - protein, 46 aa, Artificial Sequence
  Foot protein type 3 (FP-3, Mytilus galloprovincialis  mgfp-3A)
ADYYGPKYGPPRRYGGGNYNRYGRRYGGYKGWNNGWKRGRWGRKYY

SEQ ID NO: 7 - protein, 50 aa, Artificial Sequence
  Foot protein type 3 (FP-3, Mytilus edulis mefp-3F)
ADYYGPNYGPPRRYGGGNYNRYNGYGGGRRYGGYKGWNNGWNRGRRGKYW

SEQ ID NO: 8 - protein, 44 aa, Artificial Sequence
  Foot protein type 3 (FP-3, Mytilus californianus)
GAYKGPNYNYPWRYGGKYNGYKGYPRGYGWNKGWNKGRWGRKYY

SEQ ID NO: 9 - protein, 60 aa, Artificial Sequence
  partial sequence from foot protein type 4 (Mytilus californianus)
GHVHRHRVLHKHVHNHRVLHKHLHKHQVLHGHVHRHQVLHKHVHNHRVLHKHLHKHQVLH

SEQ ID NO: 10 - protein, 75 aa, Artificial Sequence
  Foot protein type5 (FP-5, Mytilus edulis)
SSEEYKGGYYPGNAYHYHSGGSYHGSGYHGGYKGKYYGKAKKYYYKYKNSGKYKYLKKARKYHRKGYKKYYGGSS

SEQ ID NO: 11 - protein, 76 aa, Artificial Sequence
  Foot protein 5 (FP-5, Mytilus edulis)
SSEEYKGGYYPGNTYHYHSGGSYHGSGYHGGYKGKYYGKAKKYYYKYKNSGKYKYLKKARKYHRKGYKKYYGGGSS

SEQ ID NO: 12 - protein, 71 aa, Artificial Sequence
  Foot protein 5 (FP-5, Mytilus coruscus)
YDDYSDGYYPGSAYNYPSGSHWHGHGYKGKYYGKGKKYYYKFKRTGKYKYLKKARKYHRKGYKKHYGGSSS

SEQ ID NO: 13 - protein, 76 aa, Artificial Sequence; identical to SEQ ID NO: 11
  mussel adhesive protein foot protein type5 from Mytilus galloprovincialis

SEQ ID NO: 14 - protein, 99 aa, Artificial Sequence
  mussel adhesive protein foot protein type 6
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 15 - protein, 194 aa, Artificial Sequence
  hybrid mussel adhesive protein (FP-151, MEFP-5 based)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 16 - protein, 196 aa, Artificial Sequence
  hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 17 - protein, 192 aa, Artificial Sequence
  hybrid mussel adhesive protein (FP-151, MCFP-5 based)
AKPSYPPTYKAKPSYPPTYKAKPSYPPTYKAKPSYPPTYKAKPSYPPTYKAKPSYPPTYKYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 18 - protein, 177 aa, Artificial Sequence
  hybrid mussel adhesive protein (FP-131)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKL

SEQ ID NO: 19 - protein, 180 aa, Artificial Sequence
  hybrid mussel adhesive protein (FP-251)
MEVHACKPNPCKNNGRCYPDGKTGYKCKCVGGYSGPTCACSSEEYKGGYYPGNSNHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKK

SEQ ID NO: 20 - protein, 182 aa, Artificial Sequence
  hybrid mussel adhesive protein (FP-353)
GCRADYYGPKYGPPRRYGGGNYNRYGGSRRYGGYKGWNNGWKRGRWGRKYYEFEFYDGYSDGYYPGSAYNYPSGSHGYHGHGYKGKYYGKGKKYYYKYKRTGKYKYLKKARKYHRKGYKKYYGGGSSGCRADYYGPKYGPPRRYGGGNYNRYGGSRRYGGYKGWNNGWKRGRWGRKYYEFEF

SEQ ID NO: 21 - protein, 309 aa, Artificial Sequence
  hybrid mussel adhesive protein (FP-13151)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKLYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 22 - protein, 9 aa, Artificial Sequence
  Fibronectin derived peptide (SPPRRARVT)
SPPRRARVT

SEQ ID NO: 23 - protein, 12 aa, Artificial Sequence
  laminin derived peptide (TWYKIAFQRNRK)
TWYKIAFQRNRK

SEQ ID NO: 24 - protein, 12 aa, Artificial Sequence
  laminin derived peptide (KNSFMALYLSKG)
KNSFMALYLSKG

SEQ ID NO: 25 - protein, 12 aa, Artificial Sequence
  collagen derived peptide (TAGSCLRKFSTM)
TAGSCLRKFSTM

SEQ ID NO: 26 - protein, 9 aa, Artificial Sequence
  vitronectin derived peptide (FRHRNRKGY)
FRHRNRKGY

SEQ ID NO: 27 - protein, 16 aa, Artificial Sequence
  Anti-microbacterial peptide (KLWKKWAKKWLKLWKA)
KLWKKWAKKWLKLWKA

SEQ ID NO: 28 - protein, 11 aa, Artificial Sequence
  Anti-microbacterial peptide (FALALKALKKL)
FALALKALKKL

SEQ ID NO: 29 - protein, 12 aa, Artificial Sequence
  Anti-microbacterial peptide (ILRWPWWPWRRK)
ILRWPWWPWRRK

SEQ ID NO: 30 - protein, 12 aa, Artificial Sequence
  Anti-microbacterial peptide (AKRHHGYKRKFH)
AKRHHGYKRKFH

SEQ ID NO: 31 - protein, 6 aa, Artificial Sequence
  collagen peptide (GLPGER)
GLPGER

SEQ ID NO: 32 - protein, 6 aa, Artificial Sequence
  collagen derived peptide (GFPGER)
GFPGER

SEQ ID NO: 33 - protein, 6 aa, Artificial Sequence
  collagen derived peptide (KGHRGF)
KGHRGF

SEQ ID NO: 34 - protein, 4 aa, Artificial Sequence
  collagen derived peptide (DEGA)
DGEA

SEQ ID NO: 35 - protein, 13 aa, Artificial Sequence
  collagen derived peptide (GEFYFDLRLKGDK)
GEFYFDLRLKGDK

SEQ ID NO: 36 - protein, 15 aa, Artificial Sequence
  collagen derived peptide (TAIPSCPEGTVPLYS)
TAIPSCPEGTVPLYS

SEQ ID NO: 37 - protein, 13 aa, Artificial Sequence
  laminin derived peptide (RQVFQVAYIIIKA)
RQVFQVAYIIIKA

SEQ ID NO: 38 - protein, 5 aa, Artificial Sequence
  laminin derived peptide (IKVAV)
IKVAV

SEQ ID NO: 39 - protein, 12 aa, Artificial Sequence
  laminin derived peptide (NRWHSIYITRFG)
NRWHSIYITRFG

SEQ ID NO: 40 - protein, 12 aa, Artificial Sequence
  laminin derived peptide (RKRLQVQLSIRT)
RKRLQVQLSIRT

SEQ ID NO: 41 - protein, 7 aa, Artificial Sequence
  laminin derived peptide (RYVVLPR)
RYVVLPR

SEQ ID NO: 42 - protein, 5 aa, Artificial Sequence
  laminin derived peptide (YIGSR)
YIGSR

SEQ ID NO: 43 - protein, 12 aa, Artificial Sequence
  laminin derived peptide (KAFDITYVRLKF)
KAFDITYVRLKF

SEQ ID NO: 44 - protein, 10 aa, Artificial Sequence
  laminin derived peptide (RNIAEIIKDI)
RNIAEIIKDI

SEQ ID NO: 45 - protein, 6 aa, Artificial Sequence
  fibronectin derived peptide (KLDAPT)
KLDAPT

SEQ ID NO: 46 - protein, 3 aa, Artificial Sequence
  fibronectin derived peptide peptide (RGD)
RGD

SEQ ID NO: 47 - protein, 6 aa, Artificial Sequence
  fibronectin derived peptide peptide (GRGDSP)
GRGDSP

SEQ ID NO: 48 - protein, 8 aa, Artificial Sequence
  fibronectin derived peptide peptide (WQPPRARI)
WQPPRARI

SEQ ID NO: 49 - protein, 15 aa, Artificial Sequence
  fibronectin derived peptide peptide (KNNQKSEPLIGRKKT)
KNNQKSEPLIGRKKT

SEQ ID NO: 50 - protein, 4 aa, Artificial Sequence
  fibronectin derived peptide peptide (REDV)
REDV

SEQ ID NO: 51 - protein, 16 aa, Artificial Sequence
  active domain from acidic fibroblast growth factor (FGF-1)
TGQYLAMDTDGLLYGS

SEQ ID NO: 52 - protein, 14 aa, Artificial Sequence
  active domain from acidic fibroblast growth factor (FGF-1)
WFVGLKKNGSCKRG

SEQ ID NO: 53 - protein, 8 aa, Artificial Sequence
  active domain from basic fibroblast growth factor (FGF-2) (FLPMSAKS)
FLPMSAKS

SEQ ID NO: 54 - protein, 16 aa, Artificial Sequence
  active domain from basic fibroblast growth factor (FGF-2)
ANRYLAMKEDGRLLAS

SEQ ID NO: 55 - protein, 14 aa, Artificial Sequence
  active domain from basic fibroblast growth factor (FGF-2)
WYVALKRTGQYKLG

SEQ ID NO: 56 - protein, 15 aa, Artificial Sequence
  Anti-microbacterial peptide (KWKLFKKIGAVLKVL)
KWKLFKKIGAVLKVL

SEQ ID NO: 57 - protein, 15 aa, Artificial Sequence
  Anti-microbacterial peptide (LVKLVAGIKKFLKWK)
LVKLVAGIKKFLKWK

SEQ ID NO: 58 - protein, 25 aa, Artificial Sequence
  Anti-microbacterial peptide (IWSILAPLGTTLVKLVAGIGQQKRK)
IWSILAPLGTTLVKLVAGIGQQKRK

SEQ ID NO: 59 - protein, 13 aa, Artificial Sequence
  Anti-microbacterial peptide (GTNNWWQSPSIQN)
GTNNWWQSPSIQN